(19) United States
(12) Patent Application Publication
(10) Pub. No.: US 2017/0042148 A1
(43) Pub. Date: Feb. 16, 2017

(54) USE OF A DICARBOXYLIC ACID TO CONTROL THE GROWTH OF HOLOPARASITIC OR HEMIPARASITIC PLANTS

(71) Applicants: UNIVERSITE CLAUDE BERNARD LYON I, Villeurbanne Cedex (FR); INSTITUT D'ENSEIGNEMENT SUPERIEUR ET DE RECHERCHE EN ALIMENTATION, SANTE ANIMALE, SCIENCES AGRONOMIQ, Marcy L'Etoile (FR); CENTRE NATIONAL DE LA RECHERCHE SCIENTIFIQUE, Paris Cedex 16 (FR); L'INSTITUT DE RECHERCHE POUR LE DEVELOPPEMENT, Marseille Cedex 02 (FR)

(72) Inventors: René BALLY, Hyeres (FR); Gilles COMTE, Villefontaine (FR); Jacques BERNILLON, Decines (FR); Floriant BELLVERT, Lyon (FR); Claire PRIGENT-COMBARET, Parmilieu (FR); Zo-Norosoa ANDRIANJAKA-CAMPS, Fully (VS) (CH); Robin DUPONNOIS, Mercurey (FR); Florence WISNIEWSKI-DYE, Mionnay (FR); Cédric BERTRAND, Perpignan (FR); Lucie MICHE, Marseille (FR)

(73) Assignees: UNIVERSITE CLAUDE BERNARD LYON I, Villeurbanne Cedex (FR); INSTITUT D'ENSEIGNEMENT SUPERIEUR ET DE RECHERCHE EN ALIMENTATION, SANTE ANIMALE, SCIENCES AGRONOMIQ, Marcy L'Etoile (FR); CENTRE NATIONAL DE LA RECHERCHE SCIENTIFIQUE, Paris Cedex 16 (FR); L'INSTITUT DE RECHERCHE POUR LE DEVELOPPEMENT, Marseille Cedex 02 (FR)

(21) Appl. No.: 15/305,673

(22) PCT Filed: Apr. 28, 2015
(86) PCT No.: PCT/FR2015/051150

§ 371 Date: Oct. 21, 2016

(30) Foreign Application Priority Data
Apr. 29, 2014 (FR) 1453891

(51) Int. Cl. A01N 37/06; C12R 1/01; A01N 63/00; C12N 1/20; A01N 37/04; A01N 37/36

(52) U.S. Cl. CPC A01N 37/06; A01N 37/04; A01N 37/36; A01N 63/00; C12N 1/20; C12R 1/01

(57) ABSTRACT

The use of a dicarboxylic acid having 2 to 5 carbon atoms to control the growth of holo- or hemiparasitic plants, as well as to the strain Azospirillum brasilense L4, registered with CNCM (Collection Nationale de Cultures de MicrOorganismes, France) under number I-4830, capable of producing a dicarboxylic acid exhibiting a bioherbicidal activity against Striga and Orobanche.

The present invention concerns the technical field of protecting agricultural crops (Poaceae, Solanaceae, Brassicaceae, Asteraceae) against root holoparasitic and hemiparasitic plants (i.e. which develop on the roots of their host plant) of the order Scrophulariales, and in particular Striga and Orobanche. More precisely, the invention concerns the use of a dicarboxylic acid comprising 2 to 5 carbon atoms or the strain Azospirillum brasilense L4 deposited at the CNCM under the number I-4830 to control the growth of holo- or hemiparasitic plants.

Holoparasitic plants are parasitic plants which do not produce chlorophyll naturally and which must thus take their organic matter from a host plant. Hemiparasitic plants are chlorophyll-producing parasitic plants which synthesize only part of the carbon-containing substances necessary to their development. These parasitic plants develop at the expense of their host by attaching to their roots via the formation of a haustorium which ends in the establishment of a connection between the conducting vessels of the xylem of the host plant and of the parasitic plant (Bouwmeester et al., 2003, Curr. Opin. Plant Biol. 6:358-364). Once connected, parasitic plants pump the sap from the host plant, slow development of the aerial parts, induce chlorosis and reduce photosynthesis (Ejeta and Butler, 2000. Parasitic plants. In: R. A. Frederiksen and G. N. Odvody (eds). Compendium of Sorghum Diseases, 2nd edition. APS Press, The American Phytopathological Society, pp. 53-56). In order to survive, these parasitic plants must produce a large quantity of seeds. Thus, once the aerial parts emerge, the parasitic plant flowers and produces enormous quantities of seeds which can remain viable in the soil for several years (Haussmann et al., 2000, Field Crop Res. 66:195-211). Plants of the genera Striga and Orobanche are members of the same order, Scrophulariales, but not of the same family (plants of the genus Striga are members of the family Scrophulariaceae and are hemiparasites, whereas plants of the genus Orobanche are members of the family Orobanchaceae and are holoparasites). These plants have many points in common: small seeds that facilitate scattering, germination dependent on the presence of strigolactone-type elicitors, trophic spoliation of the host plant via the development of a haustorium. On the other hand, they are distributed in different areas: Striga in all the tropical and subtropical regions of the world and in particular in sub-Saharan Africa, and Orobanche chiefly in the temperate countries of Asia and Central Europe and particularly the Mediterranean region. They affect the growth of a very wide variety of plants of agronomic interest (corn, rice, sorghum, millet, sunflower, rapeseed, tobacco, tomato, etc.), whether in moderate regions (Orobanche) or tropical regions (Striga). They cause substantial yield losses of a large number of crops. On the African continent: about 50 million hectares are infested by Striga, resulting in the loss of more than 10 million tons of cereals (Gressel et al., 2004, Crop Prot. 23:661-689). The yield losses of grains due to Striga infestation can vary from 5 to 100%, as a function of agroclimatic conditions, plot infestation level and cereal vulnerability (Haussmann et al., 2000, Field Crop Res. 66:195-211). In Europe, particularly in the Mediterranean basin, 16 million hectares are infested by Orobanche, with annual losses estimated at 50 million euros for sunflower, 175 million euros for tobacco, and 200 million euros for tomato (Bülbül et al., 2009, Helia 32:141-152).

The seeds of these parasitic plants are very small, which promotes their propagation over long distances by the wind, by animals, or by contaminated agricultural machinery. The transport of seeds of contaminated host plants is also a source of scattering. Since seed production by a parasitic plant is enormous (up to 100,000 seeds per plant), infested fields must usually be abandoned.

Heretofore, protecting crops against Orobanche or Striga plants has involved the use of non-selective herbicides, coupled with the use of cereal varieties resistant to these herbicides (De Groote et al., 2008, Agric. Syst. 97:83-94). These means of chemical control, besides their environmental toxicity, are costly. Moreover, in the context of the Ecophyto 2018 plan, the use of phytopharmacological products, including herbicides, will be greatly reduced.

It is also possible to stimulate the germination of Striga seeds in the absence of the host plant. As S. hermonthica is a hemiparasite, this stimulation in the absence of the host plant can lead to the death of the parasite in 2-3 days and ultimately to the reduction in the root parasite's seed reserves in the soil. This can be obtained by means of the use of ethylene (Logan and Stewart, 1991, Plant Physiol. 97:1435-1438). Ethylene is a phytohormone which can induce the germination of Striga seeds, but the application of ethylene on soil is cost prohibitive, preventing the agricultural use of this technology (Berner et al., 1999, Biological Control 15:274-282).

Other means of control exist, such as hand-pulling or the use of rotations with non-host plants, but they are less effective. The varietal selection of cereal lines resistant to plant-parasitic plants is also an alternative solution, but the costs are high.

There is, at present, no alternative control product, such as the use of naturally occurring molecules, against Striga or Orobanche available on the market.

In the literature, various studies have shown the ability of various biological agents to limit the growth of the parasitic plants Striga or Orobanche. This includes the larvae of monophagous herbivorous insects such as Phytomyza orobanchia, Eulocastra argentisparsa, fungi such as Fusarium oxysporum f.sp. orthoceras, F. oxysporum f.sp. strigae or Fusarium arthrosporioides which produce mycotoxins (i.e. fusaric acid, fumonisins and deoxynivalenol (DON)) (Amsellem et al., 2001, BioControl 46:211-228; Abuelgasim and Kroschel, 2003, FAO plant production and protection. Paper No. 120. Addendum 1, 109-144; Lendzemo et al. 2004 Field Crop Res. 91:51-61; Venne et al., 2009, Pest. Manag. Sci. 65:572-580). Mycotoxins (particularly DON) are able to inhibit the germination of Striga and Orobanche seeds, but they can trigger health problems in mammals. Particularly, they represent a risk for cattle feed and for human foodstuffs and thus cannot be regarded as completely harmless substances.

Mention may also be made of the work of Tikva Dadon et al., 2004 (Isr. J. Plant Sci. 52:83-86) titled “A factor from Azospirillum brasilense inhibits germination and radicle growth of Orobanche aegyptiaca Tikva”, describing the antagonistic activity of a metabolite produced by an Azospirillum brasilense strain on Orobanche. In this publication, the active molecule produced by this strain (the name of which is not mentioned) was identified as being an oligopeptide present in an ethanolic fraction of the supernatant. Moreover, only an activity inhibiting the germination of Orobanche seeds was shown for this strain. Furthermore, it was reported by Bouillant et al., 1997 (C. R. Acad. III Sci. Vie 320:159-162) that no Azospirillum brasilense strains have such an inhibitory activity on the germination and growth of parasitic plants. In the latter publication, it is also reported that of the two Azospirillum brasilense strains which exhibit such an inhibitory activity on the germination and growth of Striga in microcosms, only one of these strains, Azospirillum brasilense L4, exhibits a plant growth promoting effect on sorghum.

It is thus urgent, given the rapid multiplication of these parasitic plants worldwide, to develop new sustainable and environmentally-friendly solutions for controlling holo- or hemiparasitic plants, of the order Scrophulariales, which cause significant devastation of agricultural crops and plants of agronomic interest.

In this context, the present invention proposes to use a dicarboxylic acid, comprising 2 to 5 carbon atoms, to control the growth of holo- or hemiparasitic plants, and particularly to control the growth of plants of the genus Striga and/or plants of the genus Orobanche. It is also possible to use combinations of several dicarboxylic acids, comprising 2 to 5 carbon atoms, to control the growth of holo- or hemiparasitic plants, in order to obtain the desired effect.

In the context of the invention, the active agents proposed to inhibit the development of holo- or hemiparasitic plants, such as Striga or Orobanche plants, are selected from dicarboxylic acids (also referred to below as diacids) comprising 2 to 5 carbon atoms. Such diacids comprise two carboxylic acid functional groups either joined together directly, in the case of oxalic acid, or joined together by a saturated or unsaturated carbon chain comprising 1, 2 or 3 carbon atoms. This carbon chain is preferably linear and can be unsubstituted or substituted with one or more substituents, particularly of the type OH, OCH₃ and CH₂OCH₃. As examples of such diacids, mention may be made of oxalic acid, malonic acid, maleic acid, malic acid, glutaric acid and acetylene dicarboxylic acid. The use of malic acid in the L form, which was shown particularly active on the inhibition of the growth of Striga or Orobanche plants, is preferred.

Malic acid in the L form was able to be isolated, by the inventors, from the culture supernatant of a bacterial strain, Azospirillum brasilense L4, deposited, in accordance with the Budapest Treaty of 1977, under the number I-4830, on 18 Dec. 2013, at the CNCM, Collection Nationale de Cultures de Microorganismes, Institut Pasteur, 25 rue du Docteur Roux, 75724 Paris Cedex 15, France. This strain was isolated in Mali in 1995 from a sorghum field infested by a Striga parasitic plant. The strain Azospirillum brasilense L4 which was reported to have the ability to inhibit, in vitro, the growth of Striga hermontica and Orobanche ramosa and, in soil microcosms, to protect sorghum against Striga and to stimulate cereal growth (Bouillant et al., 1997, C. R. Acad. III Sci. Vie 320:159-162; Miche et al., 2000, Eur. J. Plant Pathol. 106:347-351) has now been deposited, under the number I-4830, at the CNCM (Collection Nationale de Cultures de Microorganismes, France). This strain produces in culture quite specific metabolites, compared to other Azospirillum brasilense strains (and particularly compared to that reported by Dadon et al., 2004: Isr. J. Plant Sci. 52:83-86). Indeed, in view of the noted activity, the inventors analyzed the culture supernatant of the strain Azospirillum brasilense L4, deposited under the number I-4830 at the CNCM (Collection Nationale de Cultures de Microorganismes, France), and were able to show that malic acid in the L form is responsible for the herbicidal activity noted against holo- or hemiparasitic plants. It turns out, however, that malic acid in the L form is not the major component present in the culture supernatant of the bacterial strain Azospirillum brasilense L4, deposited under the number I-4830 at the CNCM (Collection Nationale de Cultures de Microorganismes, France). The inventors also showed that this herbicidal activity noted against holo- or hemiparasitic plants could be generalized to the chemical class of dicarboxylic acids comprising 2 to 5 carbon atoms.

The invention thus proposes to use naturally occurring active agents, with low environmental impact, to limit and inhibit the growth of parasitic plants. Unlike other biological agents which act mainly by inhibiting the germination of Orobanche and Striga seeds, dicarboxylic acids comprising 2 to 5 carbon atoms are further able to inhibit the elongation of procaulomes or radicles from germinated seeds of the parasitic plant, and, consequently, to block the growth of the parasitic plant, as shown in particular from the data presented in Table 1 below. This should make it possible to decrease in a significant manner the stocks of viable seeds of holoparasites or hemiparasites in infested soil, an effect which cannot be fully obtained by the mere inhibition of the germination stage. Indeed, in the absence of conditions favorable to the germination of parasitic plants (particularly in the absence of strigolactones), the seeds can remain viable in the soil for several years.

In the context of the invention, dicarboxylic acid may be contacted with at least a seed of the holo- or hemiparasitic plant to be eradicated. Dicarboxylic acid will be used in an amount sufficient to obtain the desired effect, and particularly to block the germination of seeds and/or to inhibit the elongation of procaulomes or radicles from germinated seeds of holo- or hemiparasitic plants, of the order Scrophulariales, and particularly Striga and/or Orobanche. Preferably, 30 to 300 kg of dicarboxylic acid per hectare of treated soil can be used. If dicarboxylic acid is applied in the form of an aqueous solution, a solution comprising 1 to 20 g of dicarboxylic acid per liter of solution can be used.

Dicarboxylic acid can be applied to the seeds and/or seedlings of the plants or crops to be protected. Herbicidal compositions containing a dicarboxylic acid in accordance with the invention can notably come in the form of aqueous solutions or granules. These compositions can contain, in addition to active dicarboxylic acid, ions issuing from K₂HPO₄ or KH₂PO₄, one or more fillers such as silica, clay, kaolin or talc and one or more surfactants such as dodecylbenzene or calcium lignosulfonate. In the case of malic acid in the L form, it can be produced by chemical synthesis or directly by the bacterial strain Azospirillum brasilense L4, deposited under the number I-4830 at the CNCM (Collection Nationale de Cultures de Microorganismes, France). In this case, the acid can be isolated or the herbicidal composition can directly contain the bacterial culture supernatant. The culture supernatant can be obtained by placing the bacteria in any suitable culture medium. The growth conditions for Azospirillum brasilense bacteria known to persons skilled in the art and particularly described in Nelson and Knowles, 1978, Can J. Microbiol. 24:1395-1403, can be used. In particular, growth will be carried out at a temperature of 26 to 32° C., typically at 28° C., in a buffered aqueous medium containing salts, nutrients and a carbon source for growth of the bacterium. As examples of salts, mention may be made of NaCl, K₂HPO₄, KH₂PO₄, MgSO₄, NaCl, CaCl₂, NH₄Cl. As examples of nutrients, mention may be made of Na₂MoO₄, MnSO₄, H₃BO₃, CuSO₄, ZnSO₄, FeEDTA, yeast extract, biotin. As examples of carbon sources, mention may be made of glucose, malate and fructose in particular. As examples of such media, mention may be made of the Nfb and Nfb* media notably described in Nelson and Knowles, 1978, Can. J. Microbiol. 24:1395-1403 and Vial et al., 2006, J. Bacteriol. 188:5364-5373.

It is also possible to apply bacteria of the strain Azospirillum brasilense L4, deposited under the number I-4830 at the CNCM (Collection Nationale de Cultures de Microorganismes, France) and producing one of the active dicarboxylic acids, directly to seeds or crops of the plant to be protected or to the soil to be protected against the growth of holoparasitic and hemiparasitic plants, and thus to produce dicarboxylic acid directly in situ. Moreover, the Azospirillum brasilense strain deposited under the number I-4830 at the CNCM (Collection Nationale de Cultures de Microorganismes, France) and used in the context of the invention to inhibit the growth of holo- or hemiparasitic plants, of the order Scrophulariales, and particularly Striga and/or Orobanche, has another advantage for crop improvement since it is also able to stimulate plant growth and is able to produce a plant growth promoting agent for plants of agronomic interest (Bouillant et al., 1997, C. R. Acad. Sci. 320:159-162). Consequently, the invention proposes to use bacteria of the strain Azospirillum brasilense L4, deposited under the number I-4830 at the CNCM (Collection Nationale de Cultures de Microorganismes, France), also to produce a plant growth promoting agent for agricultural crops or plants of agronomic interest to be protected against the growth of holo- or hemiparasitic plants. The expression “plant growth promoting agent” means an agent that promotes the growth of plants of interest. Such an effect of the strain Azospirillum brasilense L4, deposited under the number I-4830 at the CNCM (Collection Nationale de Cultures de Microorganismes, France), was shown by the modification of the root system architecture of the plant of interest, and is described in the publication Bouillant et al., 1997, C. R. Acad. Sci. 320:159-162. In particular, the plant growth promoting effect can be obtained with agricultural crops or plants of agronomic interest particularly selected from corn, rice, wheat, sorghum, cow pea, tobacco, sunflower, rapeseed, cabbage, tomato, eggplant, potato, pepper, celery, bean, etc.

In particular, bacteria of the strain Azospirillum brasilense, deposited under the number I-4830 at the CNCM (Collection Nationale de Cultures de Microorganismes, France), can be used to coat seeds of such agricultural crops or plants of agronomic interest. In such a case, the bacteria are incorporated into a matrix constituting a coating for seeds of agricultural crops or plants of agronomic interest. This coating can contain one or more constituents such as peat, pearlite, gum arabic, carboxymethyl cellulose, polyvinylpyrrolidone, chitosan or alginate. The bacteria will be used in a quantity sufficient to obtain the desired effect, and particularly to block the germination of seeds of holo- or hemiparasitic plants, of the order Scrophulariales, and particularly Striga and/or Orobanche, and/or to inhibit the elongation of procaulomes or radicles from germinated seeds of holo- or hemiparasitic plants, of the order Scrophulariales, and particularly Striga and/or Orobanche, and/or to stimulate the growth of agricultural crops or plants of agronomic interest. Preferably, 3×10⁷ bacterial cells per gram of seeds of agricultural crops or plants of agronomic interest to be protected and/or to promote the growth thereof will be used.

The dicarboxylic acids comprising 2 to 5 carbon atoms proposed in the context of the invention to control the growth of holo- or hemiparasitic plants, of the order Scrophulariales, and particularly Striga and/or Orobanche, can thus be used by seed producers to coat their seeds, but also by phytopharmacology companies, both in the field of conventional agriculture (synthesis of dicarboxylic acids comprising 2 to 5 carbon atoms by chemical processes) and in the field of organic agriculture (production of active agents by living organisms, particularly in the case of malic acid in the L form).

The preparation and application examples mentioned below, in reference to the appended Figures, illustrate the invention but are in no way limiting.

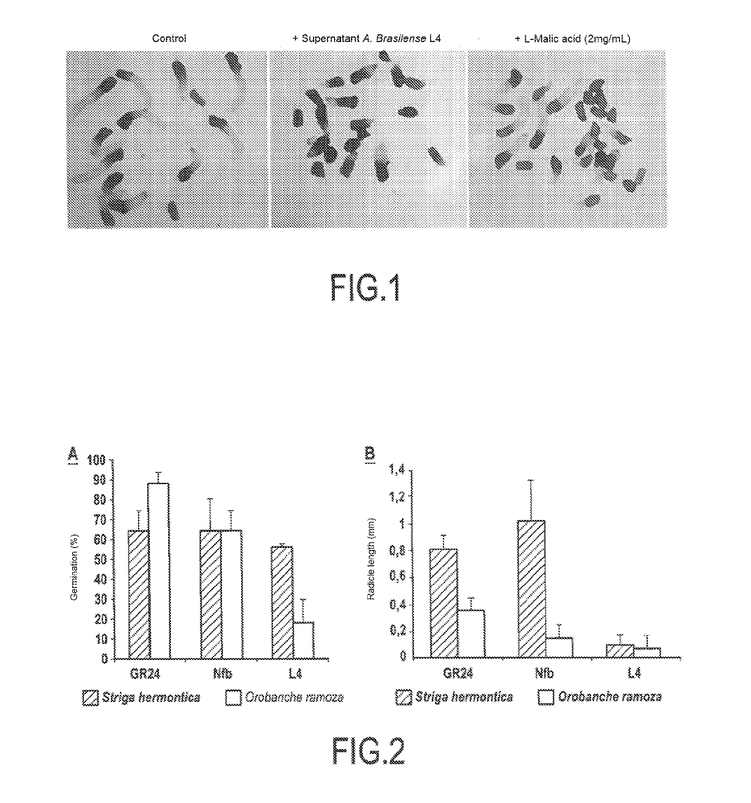

FIG. 1 presents photographs representative of the effect obtained on Striga hermontica growth, in a microtiter plate, in the presence of supernatant of the strain A. brasilense L4 grown in Nfb* medium and of L-malic acid (2 mg/mL) in phosphate buffer (50 mM).

FIG. 2 presents the effect of the supernatant of the strain A. brasilense L4 on the germination percentage of Striga hermontica and Orobanche ramosa seeds (A) and on the procaulome length of Striga hermontica and Orobanche ramosa seeds (B).

The activity of the diacids and the culture supernatant of the bacterial strain Azospirillum brasilense deposited under the number I-4830 at the CNCM (Collection Nationale de Cultures de Microorganismes, France), named L4 in the Figures, was shown in vitro and/or in soil microcosms on the parasitic plants Striga and Orobanche.

Preparation of the Products:

For the culture supernatant: culture of the strain Azospirillum brasilense L4, deposited under the number I-4830 at the CNCM (Collection Nationale de Cultures de Microorganismes, France), in Nfb* culture medium (MgSO₄, 7H₂O 100 mg/L, CaCl₂, 2H₂O 13 mg/L, NaCl 175 mg/L, Na₂MoO₄, 2H₂O 1 mg/L, MnCl₂, 4H₂O 3.5 mg/L, KH₂PO₄ 600 mg/L, K₂HPO₄ 900 mg/L, biotin 1 mg/L, Fe-EDTA 65.6 mg/L, tryptone 250 mg/L, yeast extract 125 mg/L) (Nelson and Knowles, 1978, Can J. Microbiol. 24:1395-1403, Vial et al., 2006, J. Bacteriol. 188:5364-5373) for 16 hours at 28° C.; centrifugation of the culture, recovery of the supernatant and sterilization by filtration on a 0.2 μm filter. A bioguided purification (chromatographic fractionation coupled to a biological test of the herbicidal activity of the fractions against Striga) of the supernatant of the strain A. brasilense L4 was carried out. Within the fraction exhibiting herbicidal activity, a C4 dicarboxylic acid was identified by mass spectrometry in comparison with a chemical standard. The identity of the mass spectra of the purified compound and of malic acid (chemical standard) made it possible to identify that the active molecule is malic acid. A circular dichroism detector revealed the presence of L-malic acid.

The activity of the diacids on the parasitic plants Striga and Orobanche was tested by preparing solutions of the selected diacid in water or in phosphate buffer (50 mM, pH 7) at concentrations of 1 to 20 mg/mL.

Herbicidal Effect Tested on the Holo- or Hemiparasitic Plants Striga hermontica and Orobanche ramosa:

1. In a 12-well microtiter plate, filter papers (Whatman 3) are placed at the bottom of the wells and moistened with sterile water. About 30 sterilized seeds (rinsed with 70% ethanol, then 1% (w/v) Ca(ClO)₂ and Tween® 20, then thoroughly with water) of the holo- or hemiparasitic plants are placed, in each well, on the surface of the filter papers. After 10 days of incubation at 30° C. and in the dark, increasing amounts of a diacid or of culture supernatant are added, in the presence of a germination stimulant, GR24. After 3 days of incubation at 30° C., the development of the parasitic plants in the presence of a diacid or of culture supernatant is compared to the negative control (water, phosphate buffer or sterile culture medium), with a macroscope coupled to a camera. The length of the procaulomes (radicles) of the holo- or hemiparasitic plants is measured in order to express the phytotoxic capacity of the tested products. All of the results are presented in Table 1 below.

TABLE 1
Impact of various dicarboxylic acids on Striga hermontica growth in a microtiter
plate

| | | Concentration | |
|---|---|---|---|
| | | (mg/mL of | Striga |
| Compound and formula | | buffer) | inhibition¹ |
| Buffer | — | — | − |
| Oxalic acid | 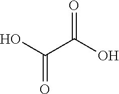 | 2 mg/mL | ++ |
| Malonic acid | 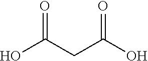 | 2 mg/mL | ++ |
| Acetylene dicarboxylic acid | 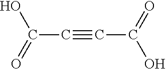 | 2 mg/mL | ++ |
| Maleic acid | 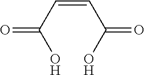 | 2 mg/mL | ++ |
| DL-Malic acid | 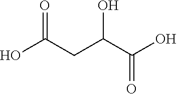 | 2 mg/mL | + |
| L-Malic acid | 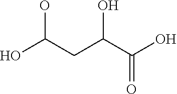 | 2 mg/mL | ++ |
| Glutaric acid | 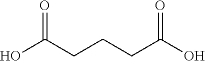 | 2 mg/mL | ++ |

¹−: no inhibition (procaulome size > 0.8 mm); +: weak inhibition (procaulome size between 0.2 and 0.8 mm); ++: strong inhibition (procaulome size < 0.2 mm)

FIG. 1 presents photographs representative of the effects obtained on Striga hermontica growth in a microtiter plate in the presence of the supernatant of the strain A. brasilense L4 grown in Nfb* and of L-malic acid (2 mg/mL) in phosphate buffer and shows their inhibitory effect on the procaulome growth of holo- or hemiparasitic plants.

FIG. 2 shows the impact of the supernatant of the strain A. brasilense L4 on the germination percentage of Striga hermontica and Orobanche ramosa seeds (A) and on the procaulome length of Striga hermontica and Orobanche ramosa seeds (B).

### Conclusion:

The ability of the culture supernatant of the bacterial strain Azospirillum brasilense L4, deposited under the number I-4830 at the CNCM (Collection Nationale de Cultures de Microorganismes, France), to stop the procaulome elongation of the holoparasitic plants Striga and Orobanche in vitro (bioassays in microtiter plates, FIGS. 1 and 2) and to inhibit Striga growth in sorghum crops in microcosms in controlled conditions (Bouillant et al., 1997, C. R. Acad. Sci. 320: 159-162) was observed. The molecules involved in this effect were isolated, by a suitable chromatographic method, then identified by mass spectrometry by comparison with chemical standards. It was also shown that various dicarboxylic acids are effective for controlling the growth of holo- or hemiparasitic plants of the order Scrophulariales, and particularly Striga and/or Orobanche (Table 1).

2. Striga hermontica seeds (about 100 seeds/pot) and sorghum seeds (1 seed/pot) were introduced simultaneously into soil mesocosms, in the presence of increasing amounts of malic acid (2 and 20 g/L) or of culture supernatant, then grown in a greenhouse or a plot.

The emergence of Striga seedlings is estimated visually, after 2 months of incubation. Phytotoxic capacity is estimated by comparison with a control without added malic acid or culture supernatant. A decrease of 22% in the number of Striga seedlings having emerged, compared to the control condition, is observed in the presence of 2 g/L malic acid and of 90% in the presence of 20 g/L malic acid. In the presence of the bacterial supernatant, a decrease of 58% in the number of Striga compared to the control condition was observed.

3. Results on Plant Growth Promoting Effect

The plant growth promoting effect of the bacterial strain Azospirillum brasilense L4, deposited under the number I-4830 at the CNCM (Collection Nationale de Cultures de Microorganismes, France), is presented in Bouillant et al., 1997 C. R. Acad. III Sci. Vie 320:159-162.

1. Use of one or more dicarboxylic acids comprising 2 to 5 carbon atoms to control the growth of holo- or hemiparasitic plants.

2. Use according to claim 1 characterized in that the dicarboxylic acid is selected from oxalic acid, malonic acid, malic acid, glutaric acid, maleic acid and acetylene dicarboxylic acid.

3. Use according to claim 1 characterized in that the dicarboxylic acid is malic acid in the L form.

4. Use according to claim 1 characterized in that the dicarboxylic acid is contacted with at least a seed of holo- or hemiparasitic plant.

5. Use according to claim 4 characterized in that the dicarboxylic acid is used in an amount sufficient to block the germination of the seed and/or to inhibit the elongation of procaulomes or radicles from the germinated seed.

6. Use according to claim 1 to control the growth of plants of the genus Striga.

7. Use according to claim 1 to control the growth of plants of the genus Orobanche.

8. Use according to claim 1 characterized in that dicarboxylic acid is applied to the seeds and/or seedlings of agronomic plants to be protected, particularly selected from corn, rice, wheat, sorghum, cow pea, tobacco, sunflower, rapeseed, cabbage, tomato, eggplant, potato, pepper, celery and bean.

9. Use according to claim 1 characterized in that the dicarboxylic acid used is produced by the strain Azospirillum brasilense L4, deposited under the number I-4830 at the CNCM (Collection Nationale de Cultures de Microorganismes, France).

10. Use according to claim 1 characterized in that the strain Azospirillum brasilense L4, deposited under the number I-4830 at the CNCM (Collection Nationale de Cultures de Microorganismes, France), is applied directly to the crops or soil to be protected against the growth of holo- or hemiparasitic plants and produces dicarboxylic acid directly in situ.

11. Use of the strain Azospirillum brasilense L4, deposited under the number I-4830 at the CNCM (Collection Nationale de Cultures de Microorganismes, France), to produce a plant growth promoting agent for agricultural crops or plants of agronomic interest to protect against the growth of holoparasitic plants, particularly selected from corn, rice, wheat, sorghum, cow pea, tobacco, sunflower, rapeseed, cabbage, tomato, eggplant, potato, pepper, celery and bean.

12. Strain Azospirillum brasilense L4, deposited under the number I-4830 at the CNCM (Collection Nationale de Cultures de Microorganismes, France).

13. Herbicidal compositions for controlling the growth of holo- or hemiparasitic plants containing a dicarboxylic acid comprising 2 to 5 carbon atoms, and ions issuing from K₂HPO₄ or KH₂PO₄, one or more fillers such as silica, clay, kaolin or talc and one or more surfactants such as dodecylbenzene or calcium lignosulfonate.

14. Compositions according to claim 13 characterized in that the dicarboxylic acid is selected from oxalic acid, malonic acid, malic acid, glutaric acid, maleic acid and acetylene dicarboxylic acid.

15. Compositions according to claim 13 characterized in that the dicarboxylic acid is malic acid in the L form.

16. Compositions according to claim 13 characterized in that they come in the form of a solution comprising 1 to 20 g of dicarboxylic acid per liter of solution.

17. Coating for seeds of agricultural crops or plants of agronomic interest comprising bacteria of the strain Azospirillum brasilense L4, deposited under the number I-4830 at the CNCM (Collection Nationale de Cultures de Microorganismes, France).

18. Coating according to claim 17 characterized in that it further comprises one or more constituents selected from peat, pearlite, gum arabic, carboxymethyl cellulose, polyvinylpyrrolidone, chitosane and alginate.
